(19) United States
(12) Patent Application Publication
(10) Pub. No.: US 2025/0375279 A1
(43) Pub. Date: Dec. 11, 2025

(54) ORAL APPLIANCE FOR DELIVERING MEDICAMENTS

(71) Applicant: R.I.P., LLC, Chatsworth, CA (US)

(72) Inventor: Rob Veis, Malibu, CA (US)

(21) Appl. No.: 18/877,130

(22) PCT Filed: Jun. 21, 2023
(86) PCT No.: PCT/US2023/025908

§ 371 Date: Dec. 19, 2024

Related U.S. Application Data
(60) Provisional application No. 63/354,196, filed on Jun. 21, 2022.

(51) Int. Cl. A61C 19/06; A61M 31/00

(52) U.S. Cl. CPC A61C 19/063; A61M 31/00; A61M 2205/0216; A61M 2205/3331; A61M 2207/00; A61M 2210/0631; A61M 2210/0637

(57) ABSTRACT

A dental appliance for delivering medicaments to teeth and gums which comprises a dental tray in which one or more recessed chambers are formed in the interior of buccal tray walls and/or lingual tray walls. The recessed chambers provide a space where a medicament can be retained when the appliance is worn by a subject.

### INTRODUCTION/BACKGROUND

Periodontal disease is an infection of gum tissues of the mouth which is generally caused by bacteria. Various treatment methods are available depending on the type and seriousness of a subject's condition, including surgery (to reduce the depth of the gingival sulcus) and systemic antibiotic treatment. However, local treatment with antibacterial or antimicrobial agents is advantageously used when more invasive or systemic treatments can be avoided or are not indicated.

Several methods have been developed for non-invasively delivering medicaments to an infected site below the gingiva in order to treat periodontal disease. One such method is the use of a dental tray for holding a medicament, such as the tray marketed under the trade name Perio Protect. Medicaments are placed in the tray, and the tray is then worn over the portion of a user's mouth where the medicament is needed. There remains a need however for improved ways to treat gum disease and to apply substances to teeth and gums topically.

### SUMMARY

The present appliance provides an improved design for delivering medicaments and other substances to teeth and gums, for example to manage periodontal disease and tooth sensitivity of a subject. The appliance comprises at least one dental tray having an anterior portion, a posterior portion, a buccal side, a lingual side, an interior portion, and an exterior portion. At least a portion of the exterior of the tray is formed from a hard plastic material, and at least a portion of the interior of the tray is formed from a soft plastic material. The dental tray includes a receptacle bounded by the interior surface of the dental tray for receiving mandibular or maxillary dentition of the subject; a buccal tray wall on the buccal side of the dental tray extending from a proximal end adjacent to the receptacle to a distal end away from the receptacle; and a lingual tray wall on the lingual side of the dental tray and extending from a proximal end adjacent to the receptacle to a distal end away from the receptacle. One or more recessed chambers are formed in one or both of the buccal tray wall and the lingual tray wall in the interior of the tray, and the recessed chambers are formed from a soft plastic material. The recessed chambers each provide a space where a medicament can be retained when the appliance is worn by a subject, and the use of a soft plastic material to form the recessed chambers allows them to be depressed in order to reduce their volume and urge a medicament present in the recess toward an area of tooth or gum of a subject. The distal ends of the buccal tray wall and the lingual tray wall intimately contact gingival tissue of the subject in order to retain a medicament in the dental tray.

The present oral appliance is adapted to deliver a medication to teeth and gums of a subject. It generally comprises a U-shaped tray having an exterior surface, an interior surface, an upper end, a lower end, a front end, a back end, a right side, and a left side. The tray includes:
a receptacle on the interior surface of the tray for receiving teeth of the subject, the receptacle having a proximal surface, a front end, a back end, a right side, a left side, a lingual side and a buccal side;
a right side buccal wall connected to the right side of the receptacle, the right side buccal wall extending apically from a proximal end to a distal end from the buccal side of the receptacle, wherein the distal end of the right side buccal wall is adapted to extend over the gums of the subject and beyond a bony height of contour of teeth adjacent to the right side buccal wall when the appliance is worn by the subject;
a right side lingual wall connected to the right side of the receptacle, the right side lingual wall extending apically from a proximal end to a distal end from the lingual side of the receptacle, wherein the distal end of the right side lingual wall is adapted to extend over the gums of the subject and beyond a bony height of contour of teeth adjacent to the right side lingual wall when the appliance is worn by the subject, wherein the right side buccal wall and right side lingual wall are connected by a right side posterior wall at the back end of the tray having a distal end;
a left side buccal wall connected to the left side of the receptacle, the left side buccal wall extending apically from a proximal end to a distal end from the buccal side of the receptacle, wherein the distal end of the left side buccal wall is adapted to extend over the gums of the subject and beyond a bony height of contour of teeth adjacent to the left side buccal wall when the appliance is worn by the subject;
a left side lingual wall connected to the left side of the receptacle, the left side lingual wall extending apically from a proximal end to a distal end from the lingual side of the receptacle, wherein the distal end of the left side lingual wall is adapted to extend over the gums of the subject and beyond a bony height of contour of teeth adjacent to the left side lingual wall when the appliance is worn by the subject, wherein the left side buccal wall and the left side lingual wall are connected by a left side posterior wall at the back end of the tray having a distal end;
a seal formed at the distal end of the receptacle, the seal extending around the right side buccal wall, the right side lingual wall, the right side posterior wall, the left side buccal wall, the left side lingual wall, and the left side posterior wall; and one or more chambers in at least one of the right side buccal wall, the right side lingual wall, the left side buccal wall, or the left side lingual wall between the seal and the proximal surface of the receptacle, each of the chambers comprising an interior surface and an interior volume for retaining the medication, the chambers being positioned adjacent to embrasure spaces of the subject's teeth when the appliance is worn by the subject.

In one embodiment, the distal end of the right side buccal wall is connected to the distal end of the right side posterior wall and the distal end of the right side posterior wall is connected to the distal end of the right side lingual wall, the distal end of the left side buccal wall is connected to the distal end of the left side posterior wall and the distal end of the left side posterior wall is connected to the distal end of the left side lingual wall, and the one or more chambers is a single chamber which extends from and fluidly communicates with the right side buccal wall, the right side posterior wall, the right side lingual wall, the left side buccal wall, the left side posterior wall, and the left side lingual wall. Preferably, the seal extends distally of the bony height of contour of the subject's teeth by between 3 mm and 7 mm, such as by about 5 mm, and the receptacle is also adapted to intimately contact occlusal surfaces of the teeth of the subject and thereby prevent a flow of the medication between the buccal wall and lingual wall on each side of the receptacle at the proximal end of the receptacle.

The chambers are preferably positioned adjacent to at least one embrasure space of the subject when the appliance is worn by the subject. The chambers are also preferably elastically deformable, and wherein applying pressure to the exterior surface of the oral urges the medication into the interior of the receptacle, such as by reducing the interior volume of the chamber and urging the medication into the interior of the receptacle. The chambers can have a depth of about 0.5 mm.

One method of forming the present oral appliance can include the steps of:

creating a model of the subject's teeth;

removing a depth of material of about 3 mm from the model at a distance of between 3 mm and 7 mm distal of the cementoenamel junction of all of the subject's teeth, wherein the material is removed from the right side buccal wall, the right side posterior wall, the right side lingual wall, the left side buccal wall, the left side posterior wall, and the left side lingual wall, thereby forming the seal;

placing a sheet of a vacuum-formable plastic material over the model; and

pulling the sheet over the model with a vacuum.

Preferably, in this method a depth of material of only about 1 mm is removed at the distal end of the right side buccal wall, the right side posterior wall, the right side lingual wall, the left side buccal wall, the left side posterior wall, and the left side lingual wall.

The present appliance can be used in a method of treating periodontal disease by placing the medication into the one or more chambers of the appliance and placing the appliance in the subject's mouth.

### BRIEF DESCRIPTION OF THE DRAWINGS

FIG. 1 is a side elevation view of an embodiment of the present appliance.

FIG. 2 is a front elevation view of the appliance of FIG. 1.

FIG. 3 is a rear perspective view of the bottom of the appliance of FIG. 1.

FIG. 4 is front perspective view of the appliance of FIG. 1.

FIG. 5 is a side elevation view of the appliance of FIG. 1 mounted on a model of a patient's dentition.

FIG. 6 is a top plan view of the appliance of FIG. 1 mounted on a model of a patient's dentition.

FIG. 7 is a rear perspective view of the bottom of another embodiment of the present appliance.

FIG. 8 is a front perspective view of the top of the appliance of FIG. 7.

FIG. 9 is a cutaway view of an illustration of a tooth and gums of a subject.

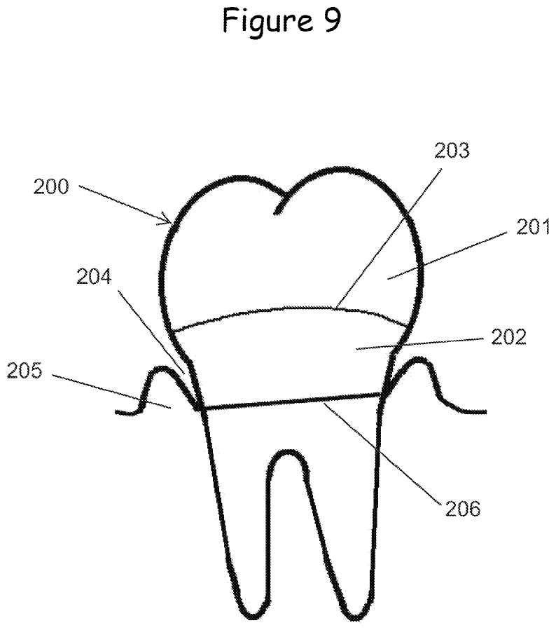

The reference numbers in the figures have the following meanings:

| Component | Subcomponent | Reference Number |
|---|---|---|
| appliance | | 10 |
| dental trays | | 15 |
| | dental tray buccal | 11 |
| | side | |
| | dental tray lingual | 13 |
| | side | |
| | dental tray right | 12 |
| | side | |
| | dental tray left side | 14 |
| | dental tray anterior | 16 |
| | portion | |
| | dental tray | 18 |
| | posterior portion | |
| | dental tray exterior | 17 |
| | surface | |
| | dental tray interior | 19 |
| | surface | |
| dental tray | | 20 |
| receptacle | | |
| | receptacle | 21 |
| | proximal surface | |
| | receptacle interior | 22 |
| | surface | |
| | front end | 23 |
| | back end | 24 |
| | right side | 25 |
| | left side | 26 |
| | lingual side | 27 |
| | buccal side | 28 |
| tray walls | | 30 |
| | buccal wall | 32 |
| | labial wall | 34 |
| | dental tray occlusal | 35 |
| | portion | |
| | proximal end | 36 |
| | distal end | 37 |
| | right side buccal | 41 |
| | wall | |
| | right side lingual | 42 |
| | wall | |
| | right side posterior | 43 |
| | wall | |
| | left side buccal wall | 51 |
| | left side lingual | 52 |
| | wall | |
| | left side posterior | 53 |
| | wall | |
| seal | | 60 |
| | retaining seal | 61 |
| | comfort seal | 62 |
| chamber or recess | | 40 |
| | interior surface | 45 |
| | interior volume | 46 |
| soft plastic | | 102 |
| material | | |
| tooth | 200 | |
| enamel | | 201 |
| cementum | | 202 |
| CEJ | | 203 |
| sulcus | | 204 |
| gingiva | | 205 |
| bony height of | | 206 |
| contour | | |
| model | | 300 |

### DETAILED DESCRIPTION

### Definitions

As used herein, the following terms and variations thereof have the meanings given below, unless a different meaning is clearly intended by the context in which such term is used.

“About” and “approximately” refer to a quantity which is within 10% of a stated quantity, preferably within 5% of the stated quantity.

“Anterior” and “front” mean in the direction of or toward or adjacent the front portion (opening) of a subject's mouth. “Back” and “posterior” mean in the direction of or toward or adjacent the rear portion of a subject's mouth.

“Apical” refers to a direction toward the root of a tooth of a user of the present appliance when the appliance is worn, i.e., toward the apex of the tooth.

“Bony height of contour” refers to the upper extent (in maxillary teeth) or lower extent (in mandibular teeth) of tooth tissue which is exposed and not covered by gum tissue.

“Buccal” means in the direction of or toward a subject's cheek. In relation to a subject's teeth, this refers to the side of the teeth facing the cheek.

“Chamber” refers to an enclosed space or cavity.

“Cementoenamel junction” or “CEJ” refers to an anatomical border identified on a tooth where the enamel, which covers the anatomical crown of a tooth, and the cementum, which covers the anatomical root of a tooth, meet.

“Concave” as used herein refers to an inwardly extending surface which forms an interior space. As applied to the medicament chambers of the present appliance, concave surfaces refer to inwardly extending surfaces which may or may not be curved or spherical.

“Coronal surface” refers to the biting surface of a tooth or to the corresponding surface of a dental tray, i.e. to a lower exterior surface of an upper dental tray or to an upper exterior surface of a lower dental tray.

“Dental tray” refers to a structure comprising a receptacle for receiving the maxillary or mandibular teeth of a subject, generally a U-shaped structure.

“Downward” and “downwardly” mean in the direction of or toward a lower portion of a subject's body. “Upward” and “upwardly” mean in the opposite direction, i.e. in the direction of or toward an upper portion of a subject's body.

“Embrasure space” refers to the generally V-shaped spaces between adjacent teeth, i.e. the area adjacent to the interproximal contact area between tooth and gum. In subjects with receding gums, the embrasure space may include a sulcus and the CEJ of a tooth.

“Hard plastic” refers to a polymer material which is not elastically deformable, such as poly(methyl methacrylate) (also known as acrylic), polycarbonate, acrylonitrile butadiene styrene (ABS) and others. The hard plastics used for the present appliance are non-toxic and compatible for use in a human subject's mouth. Hard plastics used in orthodontic applications are known to the art and can be used in the present appliance.

“Horizontal,” refers to a plane or direction which is approximately perpendicular to the sagittal and/or the coronal plane of a subject, and/or to a plane or direction which is approximately perpendicular to a surface on which a subject is supported.

“Labial” means in the direction of, toward, or adjacent to a subject's lips. In relation to a subject's teeth, this refers to the side of the front teeth facing the lips.

“Lateral” refers to a position at or toward a left side or left side of the present appliance.

“Lingual” means in the direction of, toward, or adjacent to a subject's tongue. In relation to a subject's teeth, this refers to the side of the teeth facing the tongue.

“Lower” refers to the relative position of a component in the present appliance which is closer to or toward a lower portion of a subject's body when the component is being used.

“Mandibular” refers to the lower jaw.

“Mandibular dentition” refers to the teeth of the lower jaw.

“Maxillary” refers to the upper jaw.

“Maxillary dentition” refers to the teeth of the upper jaw.

“Medicament” and “medication” refer to a substance that can be used for a medical treatment or which has a therapeutic effect. In some embodiments, the substance which is placed into the receptacle of the present appliance can be a substance having a cosmetic or other non-medical use or effect. The use of such a non-therapeutic substance is understood to be included when the term “medicament” is used herein unless otherwise indicated.

“Occlusal surface” refers to the biting surface of a tooth.

“Outward” means in a direction away from an interior portion of the present appliance.

“Recess” refers to an area of the present appliance formed in an inner wall of the receptacle of the appliance which comprises a space for retaining a medicament. “Recessed” refers to surface which extends away from another surface so as to form a space having an interior volume. Recesses include an opening which provides access to the interior volume of the space.

“Soft plastic” refers to a polymer material which is elastically deformable, such as ethylene vinyl acetate (EVA). The soft plastics used for the present appliance are non-toxic and compatible for use in a human subject's mouth.

“Subject” refers to a user of the present appliance, usually a human user.

“Sulcus” refers to an area of space (or potential space) between a tooth and the surrounding gingival tissue. A relatively deeper sulcus is commonly referred to as a “pocket.”

“Upper” refers to the relative position of a component in the present appliance which is closer to or toward an upper portion of a subject's body when being used.

“Vertical” refers to a plane or direction which is perpendicular to a horizontal plane or direction.

The terms “above,” “below,” “between,” and other terms of relative position or orientation as used herein refer to a relative position of one component of the present appliance in relation to another.

The term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise. Ranges which are described as being “between” two values include the indicated values.

### Appliance

The appliance of the present invention is a device for locally delivering medicaments or other substances to the gum and tooth tissue of a subject, in particular the gingiva, cementum, dentin, and/or enamel. The present appliance 10 comprises at least one dental tray 15, which can be an upper tray fitted to a subject's maxillary dentition, a lower tray fitted to the subject's mandibular dentition or both an upper and lower tray. The trays 15 of the present device are generally U-shaped and each comprise a right side 12, a left side 14, a buccal side 13, a lingual side 15, an anterior portion 16, a posterior portion 18, an exterior surface 17, an interior surface 19, and a tooth-receiving receptacle 20 formed on one horizontal side of the tray 15 to fit over a subject's dentition.

The receptacle 20 is configured to receive the teeth of a subject and to contact teeth on an interior surface 22. The receptacle includes an interior surface 22, a front end 23, a back end 24, a right side 25, a left side 26, a lingual side 27, and a buccal side 28. The surface adapted to contact the occlusal surfaces of a subject's teeth is referred to herein as the proximal surface 21.

The tray further comprises lateral walls 30 extending from a proximal portion 36 adjacent the receptacle 20 apically to a distal portion 37 which extends over a user's gum tissue when the appliance 10 is worn by a user. Each dental tray 15 includes a buccal wall 32 and a lingual wall 34, so as to cover the buccal and lingual sides of the subject's teeth and gums. Specifically, the present appliance 10 includes a right side buccal wall 41, a right side lingual wall 42, a right side posterior wall 43 in the posterior portion of the right side of the tray, a left side buccal wall 51, a left side lingual wall 52, and left side posterior wall 53 in the posterior portion of the left side of the tray. The exterior portions of the trays 15 further comprise an outer surface formed on the side of the tray opposite the receptacle 20, i.e. on the exterior of the tray 15. The interior surface 19 of the tray is also the interior surface 22 of the receptacle 20 and can be formed to conform to a subject's pre-existing dentition.

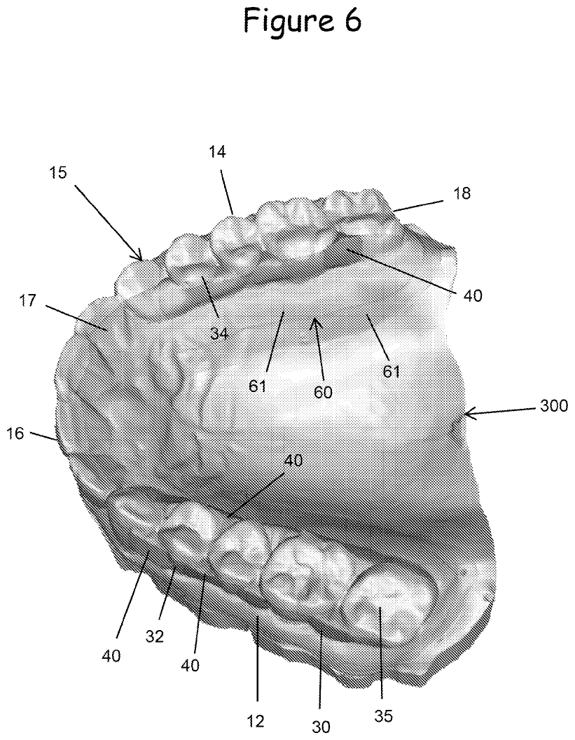

In order to deliver a medicament to a subject, in particular a liquid medicament, the medicament is placed in the interior of the tray. The side walls 30 of the tray 15 are formed so as to be close to or in contact with the teeth and gum of a subject, and the distal end 37 of the buccal wall 32 and lingual wall 34 extend over the gum line. In particular, the distal end 37 of the buccal wall 32 and lingual wall 34 each extend over the bony height of contour 206 (FIG. 6) of the teeth of a user of the present appliance 10, and are preferably in intimate contact with a user's gingival tissue along the distal extents of each of the buccal wall 32 and lingual wall 34 so as to retain a medicament within the receptacle 20 of the appliance. Preferably, the distal ends 37 of each of the buccal wall 32 and lingual wall 34 extend distally of the bony height of contour 206 of all of a user's teeth by between 3 mm and 7 mm, and the interior surfaces 19 of the buccal wall 32 and lingual wall 34 contact the surface of the user's gingiva for between 3 mm and 6 mm between the bony height of contour 206 and the distal ends 37 of the buccal and lingual walls, preferably for about 5 mm. The distal ends 37 of the buccal wall 32 and lingual wall 34 also preferably meet in the posterior portion 18 of the tray 15 and form a continuous wall structure.

In one or more areas of the interior surfaces of the tray walls 30 and the receptacle 20, one or more chambers or recesses 40 are formed. Each recess extends away from the interior surface 22 of the receptacle 20, such as to a depth of between about 3 millimeters (mm) and 7 mm, or about 5 mm, to provide a space where a medicament can be retained when the appliance 10 is worn by a subject. The chambers have an interior surface 45, which can be concave, and an interior volume 46. Chambers 40 can be located adjacent to an embrasure space between teeth of a user of the appliance.

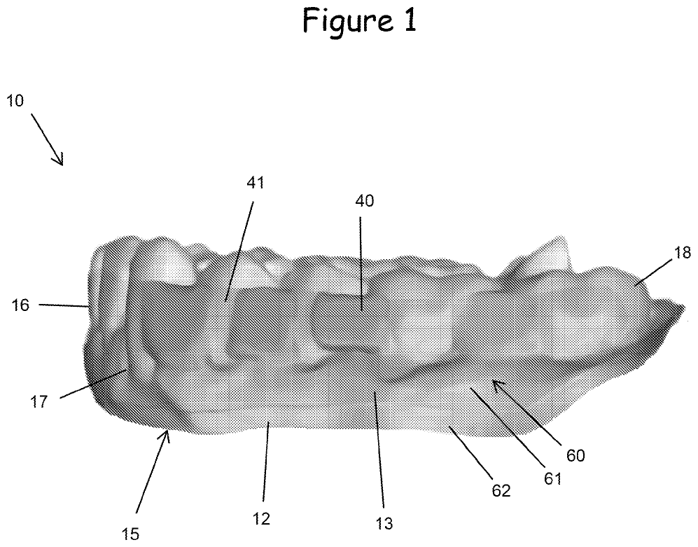

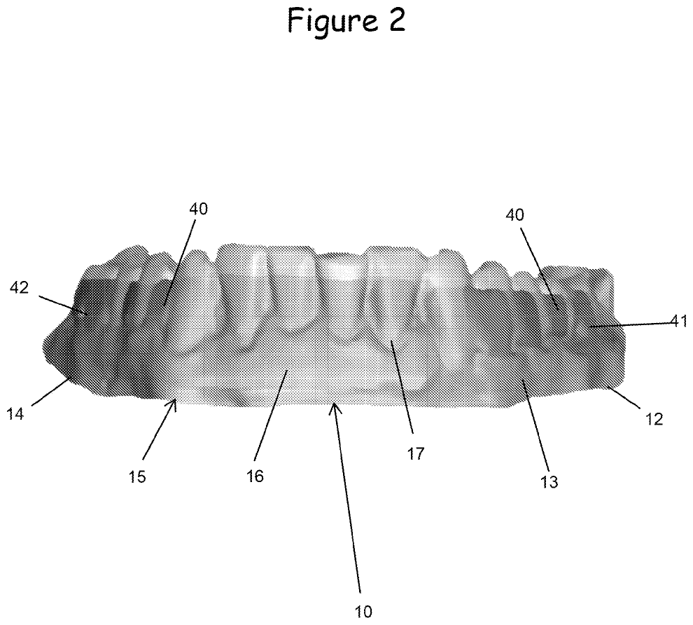

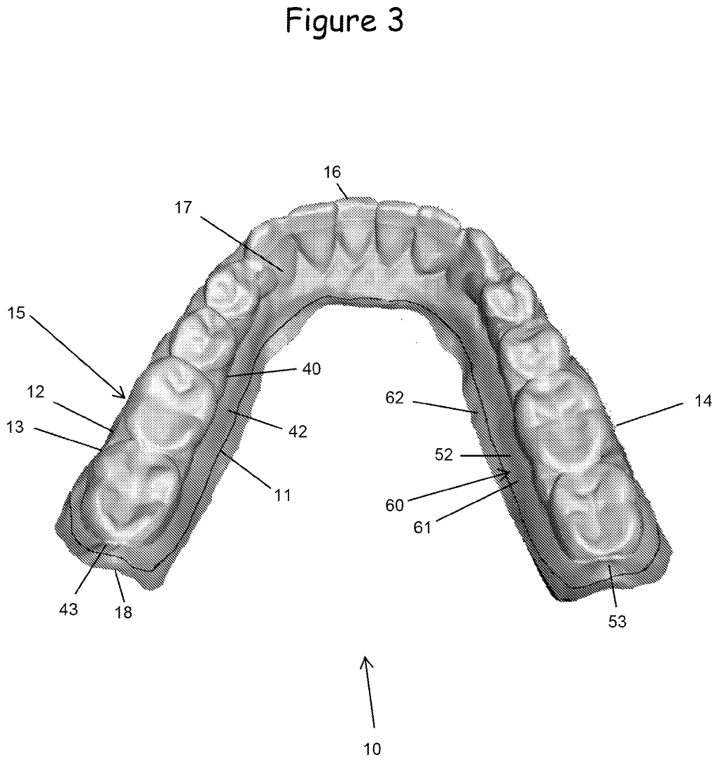

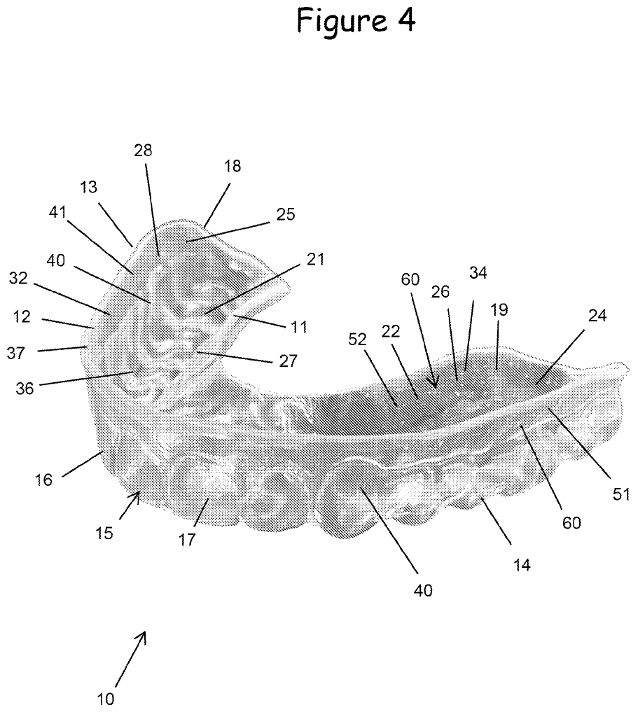

In one embodiment, shown in FIGS. 1-6, the chambers 40 of the present appliance are formed in one or more of the right side buccal wall 41, the right side lingual wall 42, the left side buccal wall 51, and the left side lingual wall 52. Preferably, chambers 40 are formed in all of these walls. In this case, the appliance includes a posterior wall at the back end of the tray on each side which joins the lingual and buccal walls, i.e., the left side buccal wall 51 and the left side lingual wall 52 are connected by a left side posterior wall 53 and the right side buccal wall 41 and right side lingual wall 42 are connected by a right side posterior wall 43. This configurations allows a seal 60 to be formed at a distal end of the walls 30 on the interior surfaces of the walls in order to better retain a medicament inside the receptacle 20 of the appliance.

The seal 60 is formed between the appliance and a subject's gums, and preferably is formed by between 3 mm and 7 mm, such as about 5 mm, above the CEJ and/or the bony height of contour of the subject's teeth when the appliance is worn. When the present appliance is formed by vacuforming, a retaining seal 61 can be formed by removing a depth of material of about 3 mm from the model at a distance of between 3 mm and 7 mm distal of the cementoenamel junction of all of the subject's teeth, where the material is removed from the right side buccal wall, the right side posterior wall, the right side lingual wall, the left side buccal wall, the left side posterior wall, and the left side lingual wall, as well as the posterior walls, i.e., a depth of material is removed from around the circumference of the interior of the receptacle 20. In this way, when the subject wears the present appliance, the tray walls will be adapted to grip the subject's gums because the gums have a greater exterior surface area than the area of the seal 60 along the interior surface of the seal. In some embodiments, the seal 60 can be placed at a distal end of the tray walls, and can extend proximally therefrom. In another embodiment, a second seal (comfort seal) 62 can be formed at the distal end of the receptacle, and the previously described retaining seal 61 can be formed proximally with respect to the comfort seal 62. The comfort seal 62 is formed by removing a depth of material of only about 1 mm at the distal end of the interior surfaces of the tray walls, i.e. the interior surfaces of the right side buccal wall, the right side posterior wall, the right side lingual wall, the left side buccal wall, the left side posterior wall, and the left side lingual wall, as well as the posterior walls, i.e., a depth of material is removed from around the circumference of the interior of the receptacle 20. The proximal surface 21 of the receptacle 20 is preferably adapted to intimately contact the occlusal surfaces of the teeth of the subject when the appliance is worn in order to inhibit or prevent a flow of the medication between the buccal wall and lingual wall on each side of the receptacle, so that a medication in the appliance is concentrated apically and around the subject's gums, for example to treat periodontal disease.

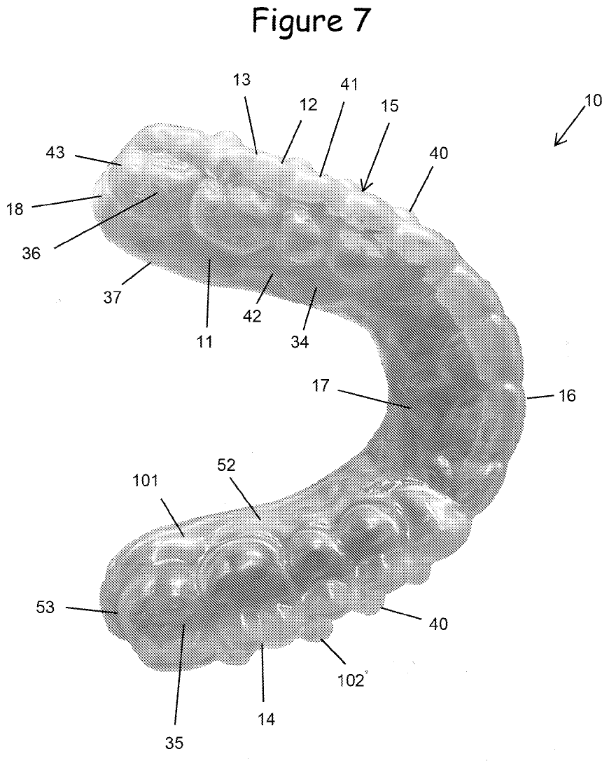

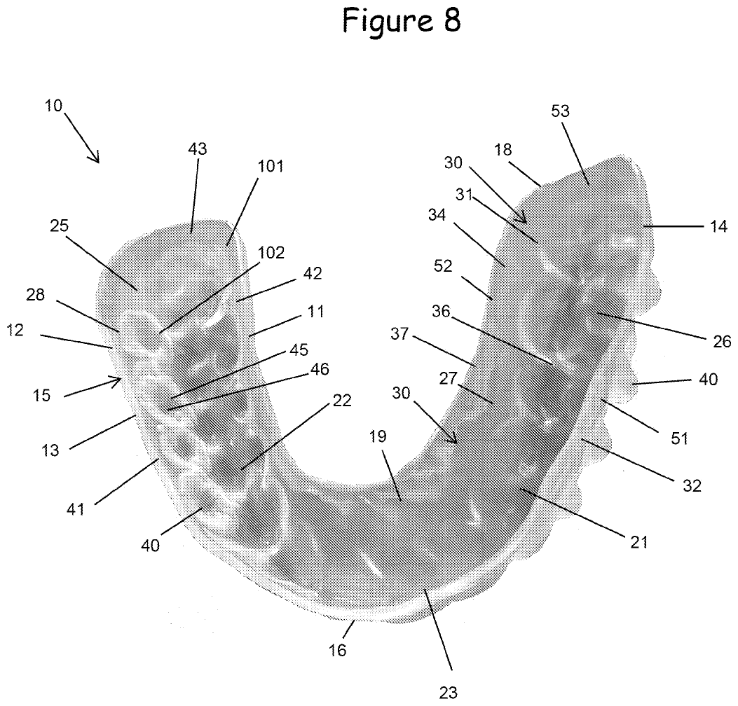

In another embodiment, shown in FIGS. 7 and 8, the recess 40 extends outwardly on the exterior surface of the tray 15, for example with a convex exterior surface 17, so as to provide a recess with a greater amount of space for retaining a medicament, and the recess is formed from an elastic, soft plastic material. In this embodiment, the outwardly extending or bulging exterior surface 17 of the recess 40 can be pressed inwardly by a subject wearing the appliance. When a medicament is present in the recess 40, such inward pressing will reduce the volume of the recess, and the medicament will be urged into the adjacent space where tooth or gum tissue to be treated is located. The recess should have sufficient elasticity to be able to be depressed by a user's finger, such as by a pressure of between 50 and 300 grams (about 0.5 to 3.0 Newtons), more preferably by a pressure of 75, 100, 150, or 200 grams (about 0.75, 1.0, 1.5, or 2.0 Newtons). In this embodiment, the dental tray 15 preferably has a laminate structure comprising a hard plastic portion 101 and a soft plastic portion 102. The receptacle(s) 40 at least should be formed from a soft plastic material as described above, and the buccal wall 32 and lingual wall 34 also preferably are formed from a soft plastic material in order to allow them to sufficiently touch or grip a user's gums to at least substantially seal a medicament within the dental tray 15. In one embodiment, the interior portions of the present appliance are formed from a soft plastic material, and some or all of the exterior portions of the dental tray 15 (except for recesses 40) are formed from a hard plastic material. The use of a hard plastic material around or adjacent to a soft plastic recess 40 allows the recess 40 to be depressed while the surround portion of the dental tray retains its structure, thereby allowing a medicament to be urged out of the recess on the interior of the tray. The distal end 37 of the buccal wall 32 and lingual wall 34 preferably extend sufficiently distally that pressure on a recess doesn't cause the distal end of the walls to deform and allow medication to be squeezed out of the dental tray's interior.

In other embodiments, the present appliance can be formed from other formable materials, typically polymer materials, known to the art. One such material is ethylene vinyl acetate (EVA), though other polymer materials compatible for oral use can be used. In one embodiment, the appliance is made from a laminate of two or more layers of material, in order to provide strength to the appliance. One such material is the vacuum forming material sold as PRO-FORM by Keystone Industries (Myerstown, PA), which is a soft EVA laminate having a thickness of 4 mm. Another such material is ERKOLOC-PRO, a bi-layer copolyester/polyurethane material having two layers and used for thermoforming.

The medicament can be in any form which can be retained in the chambers of the appliance, such as a liquid, gel, paste, other fluid. The medicament can be any substance known for treating teeth or gums, including antimicrobial agents (such as hydrogen peroxide), antibiotics, sodium fluoride (NaF), mono-fluoro-phosphate (MFP) stannous fluoride (SnF), and/or potassium nitrate (KNO₃).

### Method of Manufacture and Use

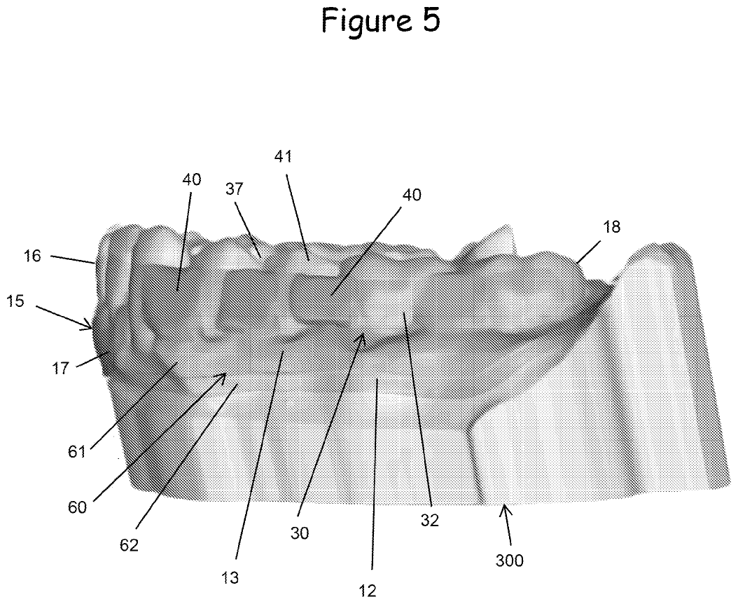

In a preferred embodiment, the present appliance is formed by vacuforming the appliance on a model 300 of a subject's teeth. After a dentist or other care provider obtains an impression of the subject's teeth, the impression is used to form a model of the subject's teeth, and a polymer material is vacuum sealed around the model, as shown for example in FIGS. 5 and 6. Vacuum forming is accomplished, for example, by clamping a sheet of plastic material into a frame, heating the material to soften it and render it pliable using a heat source, lowering the sheet of material over the model and then pulling the sheet over the model with a vacuum on the other side of the mold.

In use, a medicament is placed in the interior of the dental tray 15 of the present appliance 10, such as in the receptacle 20 and specifically in the chamber, and is then placed over the teeth and gingiva of the maxilla or mandible of a subject. If treatment of both upper and lower teeth and/or gums is desired, then an upper tray and lower tray are applied in this manner. Treatment of periodontal disease can be accomplished with the present appliance in this way, for example, through use of an appliance with chambers adjacent to any area of tooth of gum that has periodontal disease (e.g., pockets greater than 3 mm). Medicaments are circulated in the tray of the appliance and placed into contact with an affected tooth or gingival sulcus by pressing on the tray buccally or lingually and by biting into the tray, thereby deforming the elastomeric material of the appliance.

The present appliance can also be used as an occlusal surface bruxism splint, covering the maxillary and/or mandibular teeth of a subject in order to reduce nighttime clenching and to prevent tooth wear and bone loss. Bruxism can cause bone loss to occur in the presence of bacteria, so use of the present appliance to both treat infection and address bruxism can be highly beneficial to patients.

In order to provide a bruxism splint, a clinician will evaluate a subject's needs and determine an appropriate thickness for the occlusal portion 35 of the dental tray, in order to provide an appropriate amount of vertical separation between the maxillary and mandibular dentition. The exterior surface 17 of the occlusal portion 35 can be fully intercuspated, as shown in the illustrated embodiments, or can alternatively be flat, i.e. substantially planar in a horizontal orientation.

The examples set forth herein are provided to illustrate certain concepts of the disclosure. The apparatus, devices, or components illustrated above may be configured to perform one or more of the methods, features, or steps described herein. Those of ordinary skill in the art will comprehend that these are merely illustrative in nature, and other examples may fall within the scope of the disclosure and the appended claims. Based on the teachings herein those skilled in the art should appreciate that an aspect disclosed herein may be implemented independently of any other aspects and that two or more of these aspects may be combined in various ways. For example, an apparatus may be implemented or a method may be practiced using any number of the aspects set forth herein. In addition, such an apparatus may be implemented or such a method may be practiced using other structure, functionality, or structure and functionality in addition to or other than one or more of the aspects set forth herein. The various features and processes described above may be used independently of one another, or may be combined in various ways. All possible combinations and sub-combinations are intended to fall within the scope of this disclosure. In addition, certain steps or features may be omitted in some implementations. All patents, patent publications, and other publications referred to herein are incorporated by reference in their entireties.

What is claimed is:

1. An oral appliance for delivering a medication to teeth and gums of a subject comprising a generally U-shaped tray having an exterior surface, an interior surface, an upper end, a lower end, a front end, a back end, a right side, and a left side, wherein the tray comprises:
a receptacle on the interior surface of the tray for receiving teeth of the subject, the receptacle having a proximal surface, a front end, a back end, a right side, a left side, a lingual side and a buccal side;
a right side buccal wall connected to the right side of the receptacle, the right side buccal wall extending apically from a proximal end to a distal end from the buccal side of the receptacle, wherein the distal end of the right side buccal wall is adapted to extend over the gums of the subject and beyond a bony height of contour of teeth adjacent to the right side buccal wall when the appliance is worn by the subject;
a right side lingual wall connected to the right side of the receptacle, the right side lingual wall extending apically from a proximal end to a distal end from the lingual side of the receptacle, wherein the distal end of the right side lingual wall is adapted to extend over the gums of the subject and beyond a bony height of contour of teeth adjacent to the right side lingual wall when the appliance is worn by the subject, wherein the right side buccal wall and right side lingual wall are connected by a right side posterior wall at the back end of the tray having a distal end;
a left side buccal wall connected to the left side of the receptacle, the left side buccal wall extending apically from a proximal end to a distal end from the buccal side of the receptacle, wherein the distal end of the left side buccal wall is adapted to extend over the gums of the subject and beyond a bony height of contour of teeth adjacent to the left side buccal wall when the appliance is worn by the subject;
a left side lingual wall connected to the left side of the receptacle, the left side lingual wall extending apically from a proximal end to a distal end from the lingual side of the receptacle, wherein the distal end of the left side lingual wall is adapted to extend over the gums of the subject and beyond a bony height of contour of teeth adjacent to the left side lingual wall when the appliance is worn by the subject, wherein the left side buccal wall and the left side lingual wall are connected by a left side posterior wall at the back end of the tray having a distal end;
a seal formed at the distal end of the receptacle, the seal extending around the right side buccal wall, the right side lingual wall, the right side posterior wall, the left side buccal wall, the left side lingual wall, and the left side posterior wall; and
one or more chambers in at least one of the right side buccal wall, the right side lingual wall, the left side buccal wall, or the left side lingual wall between the seal and the proximal surface of the receptacle, each of the chambers comprising an interior surface and an interior volume for retaining the medication, the chambers being positioned adjacent to embrasure spaces of the subject's teeth when the appliance is worn by the subject.

2. The oral appliance of claim 1, wherein:
the distal end of the right side buccal wall is connected to the distal end of the right side posterior wall and the distal end of the right side posterior wall is connected to the distal end of the right side lingual wall,
the distal end of the left side buccal wall is connected to the distal end of the left side posterior wall and the distal end of the left side posterior wall is connected to the distal end of the left side lingual wall, and
the one or more chambers is a single chamber which extends from and fluidly communicates with the right side buccal wall, the right side posterior wall, the right side lingual wall, the left side buccal wall, the left side posterior wall, and the left side lingual wall.

3. The oral appliance of claim 1, wherein the seal extends distally of the bony height of contour of the subject's teeth by between 3 mm and 7 mm.

4. The oral appliance of claim 3, wherein the seal extends distally of the bony height of contour of the subject's teeth by about 5 mm.

5. The oral appliance of claim 1, wherein the receptacle is adapted to intimately contact occlusal surfaces of the teeth of the subject and thereby prevent a flow of the medication between the buccal wall and lingual wall on each side of the receptacle at the proximal end of the receptacle.

6. The oral appliance of claim 1, wherein at least one of the one or more chambers are positioned adjacent to at least one embrasure space of the subject when the appliance is worn by the subject.

7. The oral appliance of claim 1, wherein the one or more chambers are elastically deformable, and wherein applying pressure to the exterior surface of the oral urges the medication into the interior of the receptacle.

8. The oral appliance of claim 1, wherein applying pressure to the exterior surface of the oral appliance reduces the interior volume of the recess and urges the medication into the interior of the receptacle.

9. The oral appliance of claim 1, wherein the one or more chambers have a depth of about 0.5 mm.

10. A method of forming the oral appliance of claim 1, comprising the steps of:
creating a model of the subject's teeth;
removing a depth of material of about 3 mm from the model at a distance of between 3 mm and 7 mm distal of the cementoenamel junction of all of the subject's teeth, wherein the material is removed from the right side buccal wall, the right side posterior wall, the right side lingual wall, the left side buccal wall, the left side posterior wall, and the left side lingual wall, thereby forming the seal;
placing a sheet of a vacuum-formable plastic material over the model; and
pulling the sheet over the model with a vacuum.

11. The method of claim 10, comprising the further step of removing a depth of material of only about 1 mm at the distal end of the right side buccal wall, the right side posterior wall, the right side lingual wall, the left side buccal wall, the left side posterior wall, and the left side lingual wall.

12. A method of treating periodontal disease, comprising the steps of:
obtaining the appliance of claim 1;
placing the medication into the one or more chambers of the appliance; and
placing the appliance in the subject's mouth.
